SEQUENCE LISTING (3 sequences)

SEQ ID NO: 1 - protein, 6 aa, Mus musculus
  SITE (3)..(3): Serine at residue #3 equates to Serine491 in mouse Dab1 sequence Cdk5 phosphorylation of Serine requires a Proline (P) in the +1 position and a Lysine (K) in the +3 position
QSSPSK

SEQ ID NO: 2 - protein, 24 aa, Mus musculus
  SITE (21)..(21): Serine at Reisdue 21 equates to Serine515 in mouse Dab1 sequence Cdk5 phosphorylation of Serine requires a Proline (P) in the +1 position and a Lysine (K) in the +3 position
SSASHVSDPTADDIFEEGFESPSK

SEQ ID NO: 3 - protein, 14 aa, Mus musculus
  MOD_RES (8)..(8): PHOSPHORYLATION, equates to Serine491 in mouse Dab1 sequence Cdk5 phosphorylation of Serine requires a Proline (P) in the +1 position and a Lysine (K) in the +3 position
TPAPRQSSPSKSSA

(19) United States
(12) Patent Application Publication
(10) Pub. No.: US 2002/0172990 A1
(43) Pub. Date: Nov. 21, 2002

(54) Cyclin dependent kinase 5 phosphorylation of disabled 1 protein

(76) Inventors: Thomas Curran, Mamphis, TN (US); Lakhu Keshvara, Cordova, TN (US)

Correspondence Address: ST. JUDE CHILDREN'S RESEARCH HOSPITAL, OFFICE OF TECHNOLOGY LICENSING, 332 N. LAUDERDALE, MEMPHIS, TN, 38105, US

(21) Appl. No.: 10/078,927

(22) Filed: Feb. 19, 2002

(51) Int. Cl. G01N 33/53; G01N 33/537; G01N 33/543; C12Q 1/48

(52) U.S. Cl. 435/7.92; 435/15

(57) ABSTRACT

The present invention is based on the discovery that the Disabled 1 protein (Dab1) is a substrate for Cdk5 activity, and is selectively phosphorylated by Cdk5. An assay to determine Cdk5 activity by detection of Dab1 phosphorylation on serine amino acids that are selectively phosphorylated by Cdk5 is provided. The invention further provides an antibody and screening kit to determine Cdk5 activity, a method for detecting a neurological disorder by determining Cdk5 activity, a method of screening for compounds that increase or decrease Cdk5 activity and a method for treating a neurological disorder with such a compound.

### GOVERNMENT INTEREST

This invention was made in part with U.S. Government support under National Institutes of Health grant NIH-HS36558. The U.S. Government may have certain rights in this invention.

### FIELD OF THE INVENTION

This invention relates to the detection of kinase activity, particularly the detection of cyclin dependent kinase 5 (Cdk5) activity.

### BACKGROUND

Based upon sequence similarity, Cdk5 is classified as a member of the cyclin dependent kinase family (Lew et al., J Biol Chem 267:13383-13390, 1992; Meyerson et al., EMBO J 11:2909-2917, 1992). However, unlike other cyclin dependent kinases, Cdk5 is not involved in cell cycle regulation. Cdk5 is a serine/threonine kinase, with the active form found in differentiated neurons of the developing and mature brain. Cdk5 activity is dependent upon the association of the Cdk5 protein with a regulatory subunit, one being p35, or a proteolytic fragment of p35, known as p25. The p25 protein has a much longer half life than that of p35 and is inappropriately localized within the cell. This prolonged presence of p25 and its localization in the cytosol rather than at the plasma membrane result in constitutively active, mislocalized Cdk5 kinase activity (Patrick et al., Nature 402:615-622, 1999). Another regulator of Cdk5 is p39, which is also cleaved into a smaller more active product, p29 (Patzke and Tsai, J Biol Chem. in press).

In the developing brain, the cerebral cortex is assembled through a series of events that result in the segregation of neurons with similar properties into six layers. In the earliest phase of development, the preplate composed of Cajal-Retzius and subplate neurons, is formed between the pial surface and the ventricular zone, where cells are actively dividing. Cells exit the cell cycle in the ventricular zone and migrate radially outward towards the pial surface along glial fibers. The first-born neurons migrate past the subplate, displacing this layer away from the Cajal-Retzius cells in an area known as the marginal zone. Splitting of the preplate requires reelin, a large extracellular protein secreted by Cajal-Retzius cells. The next wave of post-mitotic neurons migrates along the same fibers, past the subplate and the older neurons in the cortical plate, before inserting beneath the marginal zone. This results in the classical inside-out pattern of the neocortex in which the sequential generation of additional layers, II-VI, occurs beneath the marginal zone (Rakic and Caviness, Neuron 14:1101-1104, 1995; Homayouni et al., Current Biology 10:R331-R334, 2000).

Mice lacking either Cdk5 or p35 protein exhibited similar abnormalities in the laminar structure of the cerebral cortex. The migrating neurons in the developing cortex of these mice were not able to by pass the cortical plate neurons to locate beneath the subplate as in normal development (Chae et al., Neuron 18:29-42, 1997; Ohshima et al., Proc Natl Acad Sci USA 93:11173-11178, 1996).

Recent studies have suggested that in addition to cortical development, Cdk5 and p35 may play a role in the outgrowth and maintenance of neuronal axons in the adult brain. Nikolic et at., Genes Dev 10:816-825, 1996, and Paglini et al., J Neurosic 18:9858-9869, 1998, have shown that Cdk5 and p35 colocalize with actin filaments in growth cones of developing neurons in cell culture. Other results show that Cdk5 kinase colocalizes with components known to be involved in regulation of focal adhesion complexes and the actin cytoskeleton (Nikolic et al., Nature 395:194-198, 1998). Cdk5 activity may also regulate other major cytoskeletal components in neurons such as intermediate filaments and microtubules (Lee and Cleveland, Annu Rev Neurosci, 19:187-217, 1996; Ohshima et al., Proc Natl Acad Sci USA 93:11173-11178, 1996; Mandelkow and Mandelkow, Trends Cell Biol 8:425-427, 1998).

Unregulated Cdk5 activity, which is associated with p25 instead of p35, has been implicated in the pathology of neurodegerative disorders, such as Alzheimer's disease (Patrick et al., supra, 1999) and amyotrophic lateral sclerosis (ALS) (Nguyen et al., Neuron, 30:135-147, 2001). Cdk5 is also implicated in regulation of numerous other cellular events including, neurite extension (Nikolic et al. Genes Development, 10:816-825, 1996), cell adhesion (Kwon et al., Current Biology 10:363-372, 2000), and axonal transport (Niethammer et al., Neuron, 28:697-711, 2000; Sasaki et al., Neuron, 28:681-696, 2000).

Cdk5 activity is tightly regulated by its binding to a regulatory subunit. Therefore in vivo Cdk5 activity cannot be determined by simply determining the amount of Cdk5 present. Nor can Cdk5 activity be determined by measuring phosphorylation of the known Cdk5 substrates because a substrate which is selectively phosphorylated by Cdk5 has heretofore not been identified. Thus there is a need for a convenient and accurate way to detect the presence of Cdk5 activity.

### SUMMARY OF THE INVENTION

The present invention is based on the discovery that the Disabled 1 protein (Dab1) is a substrate for Cdk5 activity, and is selectively phosphorylated by Cdk5 in vivo. Based on this discovery, an assay to determine Cdk5 activity by detection of Dab1 phosphorylation is provided. The assay is based on the identification of two serine amino acids in Dab1 that are selectively phosphorylated by Cdk5, serine 491 or 515, particularly serine 491.

The invention further provides an antibody and screening kit to determine Dab1 phosphorylation by Cdk5.

The invention further provides a method for detecting a neurological disorder by determining Dab1 phosphorylation by Cdk5, a method of screening for compounds that increase or decrease Cdk5 activity and a method for treating a neurological disorder with such a compound.

### DESCRIPTION OF THE SEQUENCE LISTING

SEQ ID NO:1 is a tryptic peptide that contains Dab 1 serine 491. The first amino acid in this 6 amino acid peptide corresponds to glutamine 489 of murine Dab1.

SEQ ID NO:2 is a tryptic peptide that contains Dab1 serine 515. The first amino acid in this 24 amino acid peptide corresponds to serine 495 of murine Dab1.

SEQ ID NO:3 is the portion of the Dab1 protein used as the antigen for the generation of antibodies to phosphorylated serine 491. The first amino acid in this 14 amino acid peptide corresponds to threonine 484 of murine Dab 1. A phosphate group is linked to serine 491 of the antigen, which is an amino acid in Dab1 that is selectively phosphorylated by Cdk5.

### DETAILED DESCRIPTION OF THE INVENTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Definitions

“Cdk 5” means a protein with serine/threonine kinase activity that is structurally homologous to the mitotic cyclin dependent kinases (Lew et al., J Biol Chem 267:13383-13390, 1992; Meyerson et al., EMBO J 11:2909-2917, 1992). Cdk5 proteins include, but are not limited to Cdk5 proteins cloned from human (genbank 4826674), mouse (genbank 6680907), and rat (genbank 203389).

“Cdk5 activity” means the ability of Cdk5 to phosphorylate a substrate, such as Dab 1, tau or Nudel, on serine and/or threonine in a biological sample. Cdk5 phosphorylates Dab1 on serines 491 and 515.

“Disabled 1 protein” (Dab 1) means an intracellular adapter protein that is phosphorylated by Cdk5 activity and by reelin tyrosine kinase activity. Dab1 proteins include, but are not limited to Dab1 proteins cloned from human (genbank 3288851) and mouse (genbank 1771281).

A “biological sample” may comprise a tissue, bodily fluid, blood, a cell, extract from a cell, and the like.

“Phosphorylation” means the activity of an enzyme, such as Cdk5, to add a phosphate group to an organic molecule substrate, such as Dab1. “Phosphorylated” means a substrate, such as Dab1 protein, which has a phosphate group attached to its amino acid sequence.

“Candidate sequence” means a sequence of amino acids which contains a serine followed by a proline in +1 position and a lysine in +3 position, the serine being a preferred site for Cdk5 activity (Songyang et al., Mol Cell Biol, 16:6486-6493, 1996).

“Compound” means small molecules of research or therapeutic interest; naturally-occurring factors, such as endocrine, paracrine, or autocrine factors, or factors interacting with cell receptors of all types; intracellular factors, such as elements of intracellular signaling pathways; factors isolated from other natural sources; and so forth which perturb a biological system, whether by known or unknown mechanisms and whether or not they are used therapeutically. Compounds may either stimulate or increase activity levels of a protein, or inhibit or decrease activity levels of a protein. The methods of the invention are used to identify compounds which either stimulate or inhibit Cdk5 activity in an organism or cell culture.

A “transfected cell” means a cell that has exogenous or heterologous DNA introduced into the cell. A cell may be transfected with a DNA by using any of a number of different methods well known in the art, including but not limited to electroporation, CaPO₄ precipitation, or DEAE-Dextran.

“Cell culture” refers to any in vitro culture of cells, including but not limited to continuous cell lines (e.g., with an immortal phenotype), primary cell cultures, and finite cell lines (e.g., non-transformed cells).

Antibodies having specific binding affinity for Dab1 can be produced through standard methods. As used herein, the term “antibody” includes polyclonal antibodies, monoclonal antibodies (Kohler and Milstein, Nature 256:495-497, 1975; Kozbor et al., Immunology Today 4:72, 1983; Cole et al., in Monoclonal Antibodies and Cancer Therapy, Alan R. Liss, Inc., pp. 77-96, 1985; PCT/US90/02545; Cote et al., Proc. Natl. Acad. Sci. U.S.A. 80:2026-2030, 1983), humanized or chimeric antibodies (Morrison et al., J. Bacteriol. 159-870,1984; Neuberger et al., Nature 312:604-608, 1984; Takeda et al., Nature 314:452-454, 1985), single chain antibodies (U.S. Pat. No. 4,946,778), Fab fragments, and F(ab).sub.2 fragments. Methods for producing antibodies using purified proteins or synthetic oligopeptides are well-known in the art (see Antibodies: A Laboratory Manual, Harlow and Lane, eds., Cold Spring Harbor Laboratory, New York, 1988). Antibodies may be generated against Dab1 protein produced recombinantly or isolated from tissues where Dab1 is present, and used to immunize animals, including but not limited to rabbits, mice, rats, sheep, goats, etc. Antibodies may be generated against the entire Dab1 protein or more preferably, antibodies generated against a Dab1 polypeptide. Most preferably, antibodies generated against a Dab 1 polypeptide containing serine 491 or 515, which has a phosphate group attached to serine 491 or serine 515. A peptide useful for making an antibody that detects phosphorylated serine is the peptide of SEQ ID NO:3 that contains a phosphate on serine 491.

“In vivo” means an enzymatic activity that occurs within a living organism or within a cell grown in cell culture.

“Polypeptide” means a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D or L optical isomers, and amino acid analogs and peptidomimetics.

Synthetic polypeptides, prepared using the well known techniques of solid phase, liquid phase, or peptide condensation techniques, or any combination thereof, can include natural and unnatural amino acids. Amino acids used for peptide synthesis may be standard Boc (N.sup.alpha. -amino protected N.sup.alpha. -t-butyloxycarbonyl) amino acid resin with the standard dc-protecting, neutralization, coupling and wash protocols of the original solid phase procedure of Merrifield (J. Am. Chem. Soc. 85:2149-2154, 1963), or the base-labile N.sup.alpha. -amino protected 9-fluorenylmethoxycarbonyl (Fmoc) amino acids first described by Carpino et al. (J. Org. Chem. 37:3403-3409, 1972). Both Fmoc and Boc N.sup.alpha. -amino protected amino acids can be obtained from Fluka, Bachem, Advanced Chemtech, Sigma, Cambridge Research Biochemical, Bachem, or Peninsula Labs or other chemical companies familiar to those skilled in the art. In addition, the method of the invention can be used with other N.sup.alpha.—protecting groups that are familiar to those skilled in this art. Solid phase peptide synthesis may be accomplished by techniques familiar to those in the art and provided, for example, in Stewart and Young, 1984, Solid Phase Synthesis, Second Edition, Pierce Chemical Co., Rockford, Ill.; Fields et al., Int. J. Pept. Protein Res. 35:161-214 (1990), or using automated synthesizers, such as sold by ABS. Thus, polypeptides of the invention may comprise D-amino acids, a combination of D- and L-amino acids, and various “designer” amino acids (e.g., .beta.-methyl amino acids, C.alpha.-methyl amino acids, and N.alpha.-methyl amino acids, etc.) to convey special properties. Synthetic amino acids include ornithine for lysine, fluorophenylalanine for phenylalanine, and norleucine for leucine or isoleucine. Additionally, by assigning specific amino acids at specific coupling steps, .alpha.-helices, beta. turns, beta. sheets, .gamma.-turns, and cyclic peptides can be generated. Phosphorylated polypeptides may be generated according to Perich, J., Methods in Enzymology, 201:225-233, 1991.

“Neurological disorder” means a disease or disorder of the brain and/or neurons. A neurological disorder may be a neurodegenerative disorder caused by death or loss of function of nervous tissue such disorders include but are not limited to Parkinson's disease, Huntington's disease, Alzheimer's disease, amyotrophic lateral sclerosis (ALS), stroke, epilepsy or trauma. Neurological disorder may also include disorders caused by abnormal development of the brain including but not limited to lissencephaly.

The invention discloses that Dab1 is a substrate for Cdk5 activity. Since Cdk5 activity is tightly regulated by its binding to a regulatory subunit, it has been difficult to determine Cdk5 activity by measuring the amount of Cdk5 present in a sample. Furthermore, a substrate which is selectively phosphorylated by Cdk5 had heretofore not been identified. The discovery that Dab 1 is selectively phosphorylated by Cdk5 provides a convenient assay for determining Cdk5 activity. In one aspect, the invention provides a method for detecting Cdk5 activity in a biological sample by determining whether Dab l is phosphorylated on a candidate sequence that is selectively phosphorylated by Cdk5 activity. Those biological samples which contain phosphorylated Dab 1 on the candidate sequence indicate the presence of active Cdk5 in the sample. Biological samples may be obtained from any organism, including but not limited to mouse and human, or a cell culture. Those biological samples obtained from an organism may be from any tissue or cell that contains Cdk5 activity, including but not limited to brain and blood, more specifically neurons and lymphocytes.

Biological samples may be prepared for detection of Dab1 phosphorylation on a candidate sequence using methods know by a person skilled in the art. An example is trituration of embryonic brains according to D'Arcangelo et al., Neuron, 24:471-479, 1999. The neurons obtained from the samples are either frozen or lysed immediately in lysis buffer (25 mM Tris-HCl, 1% Nonidet P-40, 150 mM NaCl, 5 mM EDTA, 1 mM sodium orthovanadate, 20 mM sodium fluoride, and 20 μg/ml each of aprotinin and leupeptin (Sigma)). Alternatively a biological sample may be prepared from whole brains which are homogenized in lysis buffer. The sample lysates are then clarified by centrifugation at 10,000× g at 4° C. for 20 minutes.

In a preferred embodiment of the above aspect, the candidate sequence contains either the serine located at position 491 or the serine located at position 515 in the amino acid sequence of Dab1, preferably the serine located at position 491. Both serine 491 and serine 515 are followed by a proline in +1 position and a lysine in +3 position making them preferred sequences for Cdk5 activity. In another embodiment the candidate sequence is the tryptic peptide of SEQ ID NO: 1 or the tryptic peptide of SEQ ID NO: 2.

In a preferred embodiment, Dab1 is phosphorylated in vivo. Detection of Dab1 phosphorylation can be accomplished with an antibody using techniques known in the art, e.g., radioimmunoassay, ELISA (enzyme-linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hem agglutination assays), complement fixation assays, immunofluorescence assays, protein A assays, immunoelectrophoresis assays, mass spectrometry, antibody array etc. Antibody binding may be determined by detecting a label on the primary antibody or by detecting binding of a secondary antibody or reagent to the primary antibody with the secondary antibody containing a label. Many means are known in the art for detecting binding in an immunoassay and are within the scope of the present invention.

Immunoprecipitation and western blotting may be performed on the sample lysates as described in Example 1 using an antibody which binds only to a serine in Dab1 which is part of a Cdk5 candidate sequence. Phosphopeptide mapping, as described in Example 1, may also be used to determine Dab1 phosphorylation. Techniques such as Western blotting, ELISA, immunocytochemistry, radiolabeling, mass spectrometry, and antibody array may be used to quantitate Dab1 phosphorylation.

In a related embodiment, Dab 1 is immunoprecipitated using antibodies which bind either a phosphorylated or non-phosphorylated form of Dab 1. In a preferred embodiment, Dab1 phosphorylation is determined by immunoblotting with an antibody which binds Dab1 only when it is phosphorylated on serine 491 or serine 515.

In another aspect of the invention, an antibody which binds Dab 1 phosphorylated on serine 491 is provided. This antibody may be generated by using the polypeptide of SEQ ID NO:3 which has a phosphate group attached to serine 491. Another aspect of the invention provides for a screening kit which contains an antibody that binds Dab1 phosphorylated on serine 491 and other reagents used in techniques suitable for detecting the binding of an antibody to Dab1. In a preferred embodiment of the above aspects, the antibody is polyclonal or monoclonal.

Screening kits containing an antibody useful for the detection of Dab1 phosphorylation on serine 491 are within the scope of the present invention. Such kits may further include one or more various components, such as for example, one or more antibodies which detect Dab1 both phosphorylated and unphosphorylated on serine 491, one or more antibodies which detect Dab1 only when it is phosphorylated on serine 491, control antibodies which do not detect Dab1 protein, etc., as will be readily apparent to those skilled in the art. Instructions, either as inserts or as labels, indicating quantities of the components to be used, guidelines for use, and/or guidelines for mixing the components, may also be included in the kit.

In another aspect, the invention provides a method for identifying a compound that regulates Cdk5 activity. This method is performed using a test sample which may consist of a tissue or blood sample which contains Cdk5 activity, a cell culture that naturally has Cdk5 activity, or a cell culture that has been transfected to express active Cdk5. Candidate compounds are added to the medium of the test sample and the level of phosphorylation of Dab1 on serine 491 or 515 of the test sample is determined and compared to a control tissue or cell culture that has received no compound.

Abnormal Cdk5 activity is characterized by an increase or decrease in Cdk5 activity in a test sample as compared to a control sample. A compound that decreases Cdk5 activity in a test sample, as compared to a control sample, indicates the ability of the compound to inhibit Cdk5 activity. A compound that increases Cdk5 activity in a test sample, as compared to a control sample, indicates the ability of the compound to increase Cdk5 activity. A compound that regulates abnormal Cdk5 activity is a therapeutic drug candidate for treating neurological disorders.

For example, one could identify a compound that regulates Cdk5 activity from a sample maintained in Dulbecco's modified Eagle's medium (BioWhitaker) supplemented with 10% fetal bovine serum (BioWhitaker), 10U/ml penicillin/straptomycin mixture (BioWhitaker), and 2 mM GlutaMAX (Lide Technologies, Inc.) by addition of a potential compound to the medium. After incubation with the potential compound the sample is lysed in lysis buffer (25 mM Tris-HCl, 1% Nonidet P-40, 150 mM NaCl, 5 mM EDTA, 1 mM sodium orthovanadate, 20 mM sodium fluoride, and 20 μg/ml each of aprotinin and leupeptin (Sigma)) and immunoprecipitated with a Dab1 antibody. The immunoprecipitates are separated by polyacrylamide gel electrophoresis and analyzed by western blotting using an antibody that binds only to Dab1 phosphorylated on serine 491.

One could also identify a compound that regulates Cdk5 activity by administering a candidate compound to an organism, then obtaining tissue or blood samples from such organism and assaying the sample for Dab1 phosphorylation on a candidate sequence using the procedure outlined above and comparing the level of Dab1 phosphorylation with the level obtained from an equivalent sample, either from the same organism prior to administration of the candidate compound or from an equivalent organism for which the candidate compound was not administered.

In a preferred embodiment, a compound identified by the methods above which inhibits or decreases Cdk5 activity may be used to treat a neurodegenerative disorder, including Parkinson's disease, Huntington's disease, Alzheimer's disease, amyotrophic lateral sclerosis (ALS), stroke, epilepsy or trauma. In another embodiment, a compound identified by the methods above which stimulates or increases Cdk5 activity may be used to treat lissencephaly and other abnormal migration defects which occur during brain development.

It has been previously shown that Dab1 is a member of the reelin pathway and this discovery that Dab 1 is also phosphorylated by Cdk5, independent of reelin, links these two parallel signaling pathways of the brain. Disruption of the reelin pathway leads to defects in several laminated structures of the brain, including the cerebral cortex, cerebellum and hippocampus (D'Arcangelo et al., J. Neurosci. Methods 82:17-24, 1998). Also, the reelin pathway has been shown to be involved in Alzheimer's Disease (Trommsdorff et al., J Biol Chem 273:33556-33560, 1998). Mice with abnormal Cdk5 activity initially appear to have normal neuronal migration, but eventually migration failures lead to inversion of layers beneath the ectopic subplate of the cerebral cortex (Ohshima et al., Proc Natl Acad Sci, 93:11173-11178, 1996; Gilmore et al. J Neurosci, 18:6370-6377, 1998). Abnormal Cdk5 activity is also associated with Alzheimer's disease and amyotrophic lateral sclerosis (ALS) (Patrick et al. Nature, 402:615-622, 1999; Nguyen et al., Neuron, 30:135-147, 2001). The abnormalities associated with defects in these two pathways suggest that Dab1 may be involved with signaling mechanisms of human neurological disorders.

The compounds identified by the methods above can be further used to modulate Cdk5 activity in a subject with a neurological disorder by administering a therapeutically effective amount of the compound. Compounds and pharmaceutical compositions suitable for use in the present invention include those wherein the active ingredient is administered to a subject in an effective amount to achieve its intended purpose. A “therapeutically effective amount” means an amount effective to prevent development of, or provide a therapeutic effect for a given condition. Determination of the effective amount is well within the capability of one with ordinary skill in the art. In the present invention the therapeutic effect is preventing and/or alleviating symptoms associated with a neurological disease by modulation of Cdk5 activity with a compound identified by the methods herein.

A “pharmaceutically acceptable carrier” refers to molecular entities and compositions that do not produce an adverse, allergic or other undesirable reaction when administered to a subject, as appropriate. Pharmaceutically acceptable carriers include any and all solvents, dispersion media, coatings, isotonic and absorption delaying agents and the like. The use of such with pharmaceutically active compounds is well known in the art. Except insofar as any conventional carrier is incompatible with the active ingredient, its use in the therapeutic compositions is contemplated. Supplementary active ingredients also can be incorporated into the compositions.

The invention also provides a method for detecting neurological disorders. The method includes (a) obtaining a biological sample from a subject suspected of having a neurological disorder, (b) determining Cdk5 activity in the biological sample by detecting phosphorylation of Dab1 on an amino acid sequence that is preferred by Cdk5 activity, and (c) comparing Dab1 phosphorylation on a Cdk5 candidate sequence with the same Dab1 phosphorylation candidate sequence in a control sample. An increase or stimulation of Dab1 phosphorylation or the decrease or inhibition of Dab 1 phosphorylation when compared to a control sample indicates the presence of a neurological disorder.

For example, the biological sample for this assay may be obtained from a biopsy of the brain which contains neurons or from a blood sample that contains lymphocytes. The control sample may be obtained from the brain or blood of a subject without a neurological disorder. The sample is lysed in lysis buffer (25 mM Tris-HCl, 1% Nonidet P-40, 150 mM NaCl, 5 mM EDTA, 1 mM sodium orthovanadate, 20 mM sodium fluoride, and 20 μg/ml each of aprotinin and leupeptin (Sigma)) and Dab1 is immunoprecipitated. Dab1 phosphorylation on serine 491 is examined by western blotting using an antibody that binds only to Dab 1 phosphorylated on serine 491.

In an embodiment of the above aspects, the biological, test, and control samples are obtained from organisms including mouse and human. Those biological, test, and control samples which are extracted from organisms are obtained from tissues including brain, specifically neurons, and blood, specifically lymphocytes. In another embodiment, the biological, test and control samples are extracted from a cell culture.

In a preferred embodiment of the method above, the invention provides for detecting neurological disorders in a subject by using an antibody that binds to Dab1 when it is phosphorylated on a candidate sequence preferred by Cdk5 activity.

The invention also provides for a method to quantitate the level of Cdk5 activity by comparing the amount of serine phosphorylated Dab1 to the total amount of Dab1 in a biological sample. Measuring the difference between the serine phosphorylated and non-phosphorylated Dab1 provides a quantitative measure of Cdk5 activity. Any method known by one skilled in the art which is capable of distinguishing phosphorylated Dab 1 from the total amount of Dab1 protein may be used. For example, equal amounts of protein from a biological sample may be immunoprecipitated with either an antibody which binds Dab1 only when it is phosphorylated on serine 491 or serine 515 or an antibody that binds to Dab1 regardless of its phosphorylation state. The Dab1 protein from each immunoprecipitate is then visualized and the amount of protein in each is measured using techniques well known in the art. Comparing the amount of protein immunoprecipitated with the antibody which binds Dab1 only when it is phosphorylated on a candidate sequence preferred by Cdk5 to the protein immunoprecipitated with the antibody that binds to Dab1 regardless of its phosphorylaton state provides a quantitative measurement of the Cdk5 activity present in a sample.

The present invention may be better understood by reference to the following non-limiting examples. These examples are presented to more fully illustrate the invention through the description of particular embodiments. These examples should in no way be construed as limiting the scope of the invention.

### EXAMPLES

### Example 1

### Cdk5 Phosphorylates Dab1 Independently of Reelin Signaling

Analysis of neurological mutant mice has led to the identification of several genes that are critical for neuronal positioning during development of the mammalian brain (Rice and Curran, Development. Annu Rev Neurosci 24:1005-1039, 2001). One of the most widely studied neurological mutants is the ataxic mouse reeler, which carries a disruption in the gene encoding Reelin, a large secreted glycoprotein (D'Arcangelo et al., Nature 374:7190723, 1995). In absence of Reelin, neurons fail to assume correct positions within the developing brain, resulting in severe defects in lamination of brain structures, including the cerebral cortex, cerebellum, and hippocampus (D'Arcangelo and Curran, Bioessays 20:235-244, 1998). Identical defects were later described in mice deficient in Dab1, or both VLDLR and ApoER2, providing strong evidence that these proteins participate in the Reelin signaling pathway (Howell et al., Nature 389:733-737, 1997; Sheldon et al., Nature 389:730-733, 1997; Trommsdorff et al., Cell 97:689-701, 1999). Mice lacking Cdk5, p35 or both p35 and p39 exhibit lamination defects that are similar but not identical to those observed in reeler (Ohshima et al., Proc Natl Acad Sci USA 93:11173-11178, 1996; Chae et al., Neuron 18:29-42, 1997; Gilmore et al., J. Neurosci 18:6370-6377, 1998; Kwon and Tsai, J Comp Neurol 395:510-522, 1998; Ko et al., J Neurosci 21:6758-6771, 2001). In contrast to the situation in reeler, initial waves of migrating neurons in Cdk5^{-/-} mice successfully split the preplate, but subsequent migration failures result in inversion of layers beneath an ectopic subplate. Therefore, Cdk5 is thought to be a component of a signaling pathway distinct from the Reelin pathway.

The phosphotyrosine-binding domain of Dab1 associates with an NPxY motif in the cytoplasmic region of VLDLR and ApoER2 (Trommsdorff et al., J Biol Chem 273:33556-33560, 1998). Reelin binds to these receptors on the surface of neurons, triggering tyrosine phosphorylation of Dab1 (D'Arcangelo et al., Neuron 24:471-479, 1999; Hiesberger et al., Neuron 24:481-489, 1999; Howell et al., Genes Dev 13:643-648, 1999). In the absence of Reelin or the receptors, Dab1 accumulates in a hypophosphorylated form (Rice et al., Development 125:3719-3729, 1998; Trommsdorff et al., Cell 97:689-701, 1999).

Tyrosine phosphorylated Dab1 may couple Reelin signaling to downstream molecular machinery involved in cell positioning. Thus, Dab1 could serve as a nexus for numerous signaling pathways.

Cdk5 is a serine/threonine kinase that is ubiquitously expressed, but its catalytic activity is dependent on the neuronal regulators p35 or p39 (Tsai et al., Development 119:1029-1040, 1993; Lew et al., Nature 371:423-426, 1994; Tsai et al., Nature 371:419-423, 1994; Tang et al., J Biol Chem 270:26897-26903, 1995). Cdk5 phosphorylates a variety of substrates in vitro, including proteins known to play a role in cell adhesion and migration (Dhavan and Tsai, Nat Rev Mol Cell Biol 2:749-759, 2001). One possible way in which Cdk5 can influence Reelin signaling is through phosphorylation of Dab1. We previously showed that Dab1 is phosphorylated predominantly on serine/threonine residues in a transient transfection system (Homayouni et al., J Neurosci 19:7507-7515, 1999). Indeed, Dab1 contains numerous potential phosphorylation sites for Cdk5. Here we show that Cdk5 phosphorylates serine 491 of Dab1 in vitro as well as in vivo. Unlike tyrosine phosphorylation, serine phosphorylation of Dab1 occurs independently of Reelin signaling. Furthermore, Cdk5 is not required for Reelin-induced tyrosine phosphorylation of Dab1. Thus, while these phosphorylation events are governed by two distinct pathways, our results raise the possibility that Cdk5 modulates Reelin signaling downstream of Dab1.

Materials and Methods

Materials.

The Dab1-hemagglutinin (Dab1-HA) expression plasmid and the GST fusion constructs containing full-length Dab1 or Dab1 domains have been described previously (Keshvara et al., J Biol Chem 276:16008-16014, 2001). Cdk5 and p35 expression plasmids were provided by Dr. Li-Huei Tsai (Harvard Medical School). Site-directed mutagenesis was conducted using the QuikChange site-directed mutagenesis kit (Stratagene, La Jolla, Calif.). Purified polyhistidine-tagged Dab1 (Dab l-His) was provided by Dr. Hee Won Park (St. Jude Children's Research Hospital). Anti-Cdk5 antibodies were obtained from Santa Cruz Biotechnology (Santa Cruz, Calif.). Rabbit anti-Dab1 antibodies (CT38) raised against the carboxyl terminal region of Dab1 and goat anti-Dab1 PTB have been described previously (Keshvara et al., J Biol Chem 276:16008-16014, 2001). B3 anti-Dab1 antibodies were a gift from Dr. Jonathan Cooper (Fred Hutchinson Cancer Research Center, Seattle).

Phosphopeptide Antibodies.

Synthesis of the phosphopeptide PSer491 (TPAPRQSS(PO₄)PSKSSA) (SEQ ID NO:3) and chemical coupling to KLH or Sepharose were carried out by Hartwell Center for Bioinformatics and Biotechnology (St. Jude Children's Research Hospital). Immunization and antibody production were carried out by Rockland (Gilbertsville, Pa.). Antisera were screened by Western blotting using lysates of HEK293T cells transfected with Dab1 and Cdk5/p35.

Antibody Purification.

To purify anti-PSer491, 250 μl of antiserum was diluted with PBS and passed several times through a column containing 500 μl of PSer491Sepharose slurry (˜5 mg of phosphopeptide). After washing the column with phosphate buffered saline (PBS), bound antibodies were eluted with 100 mM glycine, pH 2.8. The eluate was neutralized with 1 M Tris-HCl, pH 9 and dialyzed against PBS. For the purification of anti-Dab1 PTB antibodies, 10 mg of purified GST-PTB was coupled to Sepharose using AminoLink Plus Immobilization Kit (Pierce, Rockford, Ill.). Goat anti-PTB antisera were diluted with PBS and passed through this column several times, and the bound antibodies were eluted as above. The antibodies were further purified by passing once over GST-Sepharose column (Pierce).

Protein Expression.

GST fusion proteins were expressed in BL21 bacterial strains (Stratagene) as described (Keshvara et al., J Biol Chem 276:16008-16014, 2001). Briefly, bacteria were grown in LB and induced with 100μM β-D-galactopyranoside for 4 hr at 37° C. Bacterial pellets were lysed by sonicating in PBS containing 1% Triton X-100, 5 mM EDTA, 2 mM phenylmethylsulfonyl fluoride, 40 μg/ml aprotinin, and 40 μg/ml leupeptin. The lysates were incubated with glutathione-Sepharose (Amersham Pharmacia Biotech, Piscataway, N.J.) at 4° C. Bound proteins were eluted with 15 mM glutathione and dialyzed in 50 mM Tris-HCl (pH 7.5).

Animal Genotyping and Dissection.

Reeler, Cdk5-mutant, and wild-type mice were used. Conception was determined by the presence of a vaginal plug, with the day of conception considered to be embryonic day 0 (EO). Cdk5-mutant embryos and wild-type littermates were generated by crossing Cdk5 heterozygotes. Genotyping was accomplished by PCR. Embryonic and adult brains were removed by dissection and were either snap frozen in liquid nitrogen or triturated for tissue culture. Reeler embryos were generated by crossing homozygous reeler mice.

Immunohistochemical Analysis.

Embryos from E15.5-17.5 were delivered surgically and perfused intracardially with 4% paraformaldehyde in 0.1M PBS (pH 7.2). The tissue was post-fixed for four hours at 4° C., cryoprotected in 25% sucrose, embedded in TBS tissue freezing medium (Triangle Biomedical Sciences, Durham, N.C.) on dry ice and processed using standard techniques (Rice et al., Development 125:3719-3729, 1998). For all experiments, control and test tissues were embedded together in one block to minimize differences in section thickness. Colocalization of Cdk5 and Dab1 was performed using rabbit anti-Cdk5 antibodies (1:100) (Santa Cruz) and goat anti-Dab1 antibodies (1:100). Nonspecific antibody binding was blocked with 2.5% BSA in PBS (pH 7.3) containing 0.01% Triton-X 100 (PBST) for one hour at room temperature. For double staining, goat anti-Dab1 and rabbit anti-Cdk5 antibodies were diluted together in BSA-PBST and incubated with tissue sections at 4° C. overnight. Tissue sections were washed thoroughly in PBS and then incubated with AlexaFluor 594 conjugated donkey anti-goat IgG (1:200) and AlexaFluor 488 conjugated goat anti-rabbit IgG (1:200) (Molecular Probes, Eugene, Oreg.) for one hour at room temperature. Sections were washed as described above and coverslips were mounted with Vectashield Mounting Medium (Vector Labs, Burlingame, Calif.). Adjacent sections identically processed without the primary antibody did not show any significant staining above background. Embryonic day 18.5 sagittal sections from Cdk5^{+/+} and Cdk5^{-/-} brains were incubated in a humid chamber overnight at 4° C. with rabbit anti-Dab1 antibodies (B3, 1:800). Rabbit anti-Dab1 antibodies were detected using the Vectastain Elite ABC kit (Vector labs) as described previously (Rice et al., Development 125:3719-3729, 1998). Cdk5^{+/+} and Cdk5^{-/-} tissues were cut and slide mounted in the same tissue block and incubated for equivalent times in diaminobenzidine (DAB) substrate (Kirkegaard and Perry Laboratories, Gaithersburg, Md.). As negative controls, adjacent sections from the same tissue block were identically processed without the primary antibody and no DAB reaction product was observed. Slides were dehydrated and mounted by standard techniques. Immunofluorescence and immunohistochemical detection of Dab1 and Cdk5 were carried out using an Olympus BX60 upright microscope and images were acquired with a Hamamatsu C5810 video camera and imported directly into Adobe Photoshop 5.0. Contrast and brightness were applied equally to each figure to obtain high quality photomicrographs.

Reelin Stimulation and Brain Lysates.

Stimulation of embryonic neurons with purified Reelin has been described previously (Keshvara et al., J Biol Chem 276:16008-16014, 2001). Briefly, neurons obtained by trituration of embryonic brains were treated either with purified recombinant Reelin or with a control supernatant for 15 minutes at 37° C. After incubation neurons were either snap frozen or lysed immediately. Reelin-treated neuronal pellets were lysed in cell lysis buffer (25 mM Tris-HCl, 1% Nonidet P-40, 150 mM NaCl, 5 mM EDTA, 1 mM sodium orthovanadate, 20 mM sodium fluoride, and 20 μg/ml each of aprotinin and leupeptin). Whole brains were also homogenized in this lysis buffer. Cell lysates as well as brain homogenates were clarified by centrifugation at 10,000× g at 4° C. for 20 min.

Cell Culture and Transfection.

HEK293T cells (ATCC) were maintained in Dulbecco's modified Eagle's medium (BioWhitaker, Walkersville, Md.) supplemented with 10% fetal bovine serum (BioWhitaker), 10 U/ml penicillin/streptomycin mixture (BioWhitaker), and 2 mM GlutaMAX (Invitrogen, Carlsbad, Calif.). Transfections were carried out using FuGene 6 transfection reagent (Roche Molecular Biochemicals, Indianapolis). 24 hr after transfection, cells were lysed in cell lysis buffer.

Immunoprecipitation and Western Blotting.

Cell or brain lysates were incubated with anti-Cdk5 or anti-Dab1 antibodies at 4° C. for 2 hr. Immune complexes were collected with protein G-agarose beads (Pierce) and washed three times with cell lysis buffer. Immunoprecipitates were boiled in SDS sample buffer, separated by SDS-PAGE, and blotted to nitrocellulose membranes. Nitrocellulose membranes were blocked with 5% non-fat dry milk in TBST (50 mM Tris, pH 7.4, 150 mM NaCl, 1% Tween 20) for 1 hr at room temperature. The following antibodies were used for various immunoblots: anti-PSer491 (1:1000), anti-Cdk5 (1:2000), anti-PY198 (1:100), 4G10 (1:2000) (Upstate Biotechnology, Lake Placid, N.Y.), and anti-Dab1 (CT38, 1:5000). All dilutions were made in blocking solution, and incubations were carried out at room temperature for one hour. The membranes were washed in TBST and incubated with horseradish peroxidase-conjugated secondary antibody (1:10,000) for 1 hr. Detection was carried out using Super Signal West Dura Extended Duration Substrate (Pierce).

In Vitro Kinase Assay.

For kinase assays, anti-Cdk5 immunoprecipitates were washed three times with cell lysis buffer and once with 25 mM Tris-HCl, pH 7.5. The beads were then incubated with a kinase reaction mixture containing 5 μg substrate (GST fusions or Dab1-His), 10 mM MgCl₂, 10 μCi ATP (10 μM) in 25 mM Tris-HCl, pH 7.5. The reactions were carried out at 30° C. for 15 min and then stopped by boiling in SDS sample buffer. Phosphorylated proteins were separated by SDS-PAGE, transferred to nitrocellulose membranes, and detected by autoradiography.

Phosphopeptide Mapping.

Phosphoproteins were excised from nitrocellulose membranes and digested with trypsin (Promega, Madison, Wis.) as described previously (Keshvara et al., J Biol Chem 276:16008-16014, 2001). Phosphopeptides were dried and separated by electrophoresis on an alkaline 40% polyacrylamide gel (alkaline-PAGE), as described (Keshvara et al., J Biol Chem 276:16008-16014, 2001). After the tracking dye had migrated to RF=0.5, the gel was dried, and phosphopeptides were detected by autoradiography.

Results

Dab1 and Cdk5 are Coexpressed in the Developing Brain

Mice that lack Dab1 or Cdk5 exhibit defects in the migration patterns of neurons in the cortical plate and in the cerebellum. However, the defective migration of neurons in Cdk5 deficient mice is distinct from the abnormal migrations observed in Dab1-deficient mice. During early corticogenesis, Dab1 is present in the cells that occupy the ventricular zone during the preplate stage of development, and later it is present in migrating neurons in the intermediate zone and in neurons within the cortical plate (Rice et al., Development 125:3719-3729, 1998). Cdk5 is detected as early as E14.5 in postmitotic neurons in the cerebral cortex (Tsai et al., Development 119;1029-1040, 1993), and it is predominantly present in thalamocortical tracts. In P0 cerebellum, Dab1 is expressed in Purkinje cells as they migrate to form the Purkinje cell layer. Cdk5 is expressed in Purkinje cells in the cerebellum, but it is also present in large cells of the cerebellar cortex such as the basket, Lugaro or Golgi cells (Ohshima et al., J Neurosci 19:6017-6026, 1999). These analyses show that Dab1 and Cdk5 are present in overlapping cell populations in the cerebral cortex and in the Purkinje cells of the cerebellum. Therefore, it is possible that Cdk5 phosphorylates Dab1 within these cell populations.

Cdk5 Phosphorylates Dab1 in vitro

To determine if Cdk5 can phosphorylate Dab1 in vitro, an active Cdk5/p35 complex was immunoprecipitated from brain homogenates and used to phosphorylate recombinant polyhistidine-tagged Dab1 (Dab1-His). Indeed, Dab1 was highly phosphorylated by anti-Cdk5 immunoprecipitated from brain extracts of wild-type mice. In contrast, immunoprecipitates from brain extracts of Cdk5-deficient littermates did not yield any significant kinase activity.

To begin to localize the Cdk5 phosphorylation sites on Dab1, we repeated the in vitro Cdk5 kinase assays using GST fusion proteins representing three distinct regions of Dab1, namely, the PTB domain (GST-PTB; residues 1-179), the middle region (GST-Mid; 180-399), and the carboxy terminal region (GST-CT; 400-555). Fusion proteins containing the PTB domain and the middle region of Dab1 were not phosphorylated to any significant extent. In contrast, the GST fusion product containing the carboxy terminal region of Dab1 was phosphorylated to almost the same extent as full-length Dab1. Therefore, the major sites of in vitro phosphorylation by Cdk5 are likely to be confined to the carboxy terminus of Dab1. To confirm this observation, we generated tryptic phosphopeptides from in vitro phosphorylated Dab1-His and GST-CT and analyzed them by electrophoretic separation on a 40% acrylamide gel. Two major phosphopeptides were clearly resolved, and both were present in the carboxy terminal region of Dab1.

Cdk5 exhibits a strong preference for serine and threonine residues with an adjacent proline and a basic amino acid at the third residue (Songyang et al., Mol Cell Biol 16:6486-6493, 1996). Although there are several SP/TP sequences present within the carboxy terminal domain of Dab1, only two sites, serines 491 and 515, correspond to the consensus sequence preferred by Cdk5. Both serine 491 and serine 515 are followed by proline in the +1 position and lysine in the +3 position. Serine 491 is located within a small tryptic fragment QSSPSK (SEQ ID NO:1) comprised of only six amino acids, representing the smallest possible tryptic peptide that would contain a Cdk5 phosphorylation site within the carboxy terminal domain of Dab1. The other putative site for Cdk5 phosphorylation, serine 515, is contained within a much larger tryptic fragment, SSASHVSDPTADDIFEEGFESPSK, (SEQ ID NO:2). To test these fragments for Dab1 phosphorylation, the Cdk5/p35 complex was immunoprecipitated from detergent lysates of either Cdk5^{-/-} or Cdk5^{+/+} P0 brains. The immunoprecipitates were then used as a source of kinase activity to phosphorylate either full length polyhistidine-tagged Dab1 or GST fusion proteins containing Dab1 domains (GST-PTB, 1-179; GST-Mid, 180-399; GST-CT, 400-555) in the presence of [y-³²P]-ATP in vitro. Proteins were separated by SDS-PAGE, transferred to nitrocellulose, and located by autoradiography. In vitro phosphorylated GST-CT and Dabl-His were trypsinized off the membrane, and tryptic fragments were resolved by alkaline 40% polyacrylamide gel electrophoresis. Autoradiography revealed two major phosphopeptides. The carboxy terminal region of Dab1 gives rise to two phosphopeptides as a result of phosphorylation by Cdk5/p35. Two serines, serine 491 and serine 515, contain the consensus sequence for Cdk5 phosphorylation, including proline in +1 position and lysine residue in +3 position. Therefore, our phosphopeptide mapping analysis was consistent with the hypothesis that Cdk5 preferentially phosphorylated serines 491 and 515 in vitro.

Anti-PSer491 Antibodies Recognize Cdk5-Catalyzed Dab1 Phosphorylation

We recently used phosphopeptide-specific antibodies to identify Reelin-induced sites of tyrosine phosphorylation in Dab1 (Keshvara et al., J Biol Chem 276:16008-16014, 2001). Therefore, we adopted the same approach to determine whether Dab1 is phosphorylated by Cdk5 in vivo. While our phosphopeptide mapping analysis suggested that both serines 491 and 515 are in vitro sites of Cdk5 phosphorylation, we consistently observed that serine 491 was more highly phosphorylated. Therefore, we chose to generate antibodies against a phosphopeptide corresponding to this phosphorylation site. The efficacy of the resulting antisera was determined by Western blotting using lysates from 293T cells transiently transfected with either Dab1 alone or in the presence of Cdk5 and p35. The antiserum preferentially recognized Dab1 when it was co-transfected with Cdk5 and p35, suggesting that the antibodies specifically reacted with phosphorylated Dab1. To confirm the specificity of the antibodies, we generated a mutant of Dab1 in which serine 491 was substituted with an alanine residue (491A). The antibodies also failed to recognize this mutant protein as well as a combination mutant in which both serine 491 and serine 515 of Dab1 were converted to alanine (AA). In contrast, substitution of serine 515 alone (515A) with alanine did not have any effect on antibody recognition. Western blots with anti-Dab1 antibodies confirmed that the mutant forms of Dab1 were expressed at equivalent levels. Therefore, these results indicate that Cdk5 phosphorylates Dab1 on serine 491 in transfected cells, and that the antibodies are highly specific for this phosphoserine site. Cdk5-dependent in vivo phosphorylation of Dab1 on serine 491 To determine whether serine 491 is phosphorylated in vivo, Dab1 was immunoprecipitated from brain extracts prepared from E16.5 Cdk5^{-/-}, Cdk5^{+/-}, or Cdk5^{+/+} embryos, and Western blots were carried out using anti-PSer491 antibodies. Serine 491 was phosphorylated to the same extent in brain extracts from Cdk5^{+/-} and Cdk5^{+/+}. In contrast, phosphorylation of this serine was not detected in brain extracts from Cdk5-deficient mice, suggesting that in vivo phosphorylation of serine 491 of Dab1 is catalyzed by Cdk5. Since Cdk5-deficient mice exhibit neuronal migration defects similar to those observed in reeler, we asked whether the absence of Cdk5 had any effect on Reelin signaling. Previously we demonstrated that tyrosine 198 of Dab1 is specifically phosphorylated in response to Reelin (Keshvara et al., J Biol Chem 276:16008-16014, 2001). Therefore, we used antibodies specific for this phosphotyrosine (anti-PY198) to investigate Reelin signaling in Cdk5-deficient mice. Immunoblots with anti-PY198 antibodies revealed only a modest decrease in phosphorylation of tyrosine 198 in Cdk5^{-/-} mice. A characteristic hallmark of a failure in Reelin signaling is the accumulation of Dab1 (Rice et al., Development 125:3719-3729, 1998). Indeed, we observed a slight increase in Dab 1 protein levels in lysates from Cdk5^{-/-} brains. Western analysis with anti-actin antibodies confirmed equal sample loading. Thus, these results suggest that there is a slight but detectable defect in Reelin signaling in Cdk5^{-/-} brains at E16.5.

At P0, the cortical plate is largely populated by neurons that have completed migration in wild-type mice. To determine if Reelin signaling is intact at this time in Cdk5^{-/-} mice, we repeated the above experiments using lysates from P0 Cdk5^{-/-}, Cdk5^{-/+}, and Cdk5^{+/+} brains. As before, we did not observe any phosphorylation of Dab1 on serine 491 in Cdk5^{-/-} mice. In contrast to the situation at E16.5, tyrosine phosphorylation of Dab1 was not detected at P0, suggesting that the Reelin signaling pathway is severely compromised at this age in Cdk5^{-/-} mice. Indeed, we also observed a clear accumulation of Dab1 (3-fold) in Cdk5^{-/-} mice at P0. In addition, the electrophoretic mobility of Dab1 from Cdk5^{-/-} mice was distinct from that observed in extracts from wild-type mice. This mobility shift was most likely a consequence of hypophosphorylation of Dab1 on both tyrosine and serine residues. Taken together, these results suggest that in Cdk5^{-/-} mice, the Reelin signaling pathway is intact during the initial stages of cortical plate formation. However, as the brain develops, Reelin signaling is progressively impaired in the absence of Cdk5.

Dab1 Levels are Elevated in Ectopically Positioned Neurons in the Cerebral Cortex and Cerebellum

Elevated levels of Dab1 are detected in ectopic neurons in reeler mice (Rice et al., Development 125:3719-3729, 1998). To determine whether the elevated Dab1 levels detected in Cdk5^{-/-} mice occur specifically in ectopic neurons, we analyzed the distribution of Dab1 in these mice by immunohistochemistry. Immunohistochemistry was performed using rabbit antibodies to Dab1 (B3) on sagittal tissue sections from E18.5 littermates generated by mating Cdk5^{+/-} mice. In wild-type mice, the cortical plate is positioned above the subplate, and it is organized into tightly packed layers of radially oriented neuronal cell bodies. In this case, Dab1 is localized to the cell bodies and the apical dendrites of postmitotic neurons in the cortical plate. In Cdk5^{-/-} mice, the cerebral cortex develops into an early cortical plate separated from an ectopic cortical plate located underneath the subplate. Immunostaining for Dab1 demonstrated that the ectopic cortical plate contained increased levels of Dab1 compared to the normally positioned cortical plate. These differences in Dab1 levels were observed at all levels from lateral to dorsal cerebral cortex.

In the wild-type cerebellum at E18.5, Purkinje cells migrate to form a Purkinje cell layer at superficial positions beneath the external germinal layer. Dab1 is expressed in Purkinje cells while they are still migrating and in cells that occupy the Purkinje cell layer. Interestingly, the levels of Dab1 are slightly elevated in small populations of Purkinje cells caught in the process of migrating into the Purkinje cell layer. These populations reside in sub-compartments of the developing cerebellum that have been described previously (Nunzi et al., J Comp Neurol 404:97-113, 1999; Ozol et al., J Comp Neurol 412:95-111, 1999). In the Cdk5^{-/-} cerebellum, Purkinje cells fail to migrate out of deep locations, and instead they accumulate in ectopic positions near the neuroepithelium. Dab 1 levels are dramatically elevated in these ectopically located Purkinje cells in the mutant cerebellum. Together, these histological studies demonstrate that in the absence of Cdk5, Dab1 levels are elevated in specific populations of neurons located in ectopic positions in the cerebral cortex and the cerebellum, suggesting that the Reelin signaling pathway is compromised in these cells.

Cdk5-Deficient Neurons Can Respond to Reelin

Since the accumulation of hypophosphorylated Dab1 in Cdk5^{-/-} neurons implies that Reelin signaling is defective, we sought to determine whether the defect in Reelin signaling is intrinsic to Cdk5-deficient neurons or whether it arises as an indirect consequence of the ectopic location of these neurons. Neurons isolated from E16.5 Cdk5^{-/-} or Cdk5^{+/+} embryos were suspended in DMEM alone (Con) or Reelin-enriched DMEM (Reln). Treated neurons were then lysed and immunoprecipitated with anti-Dab 1 antibodies. The immunoprecipitates were separated by SDS-PAGE and transferred to nitrocellulose, and Western blots were performed using 4G10 anti-phosphotyrosine (PTyr) or anti-Dab1 (Dab1) antibodies. Cdk5-deficient neurons clearly responded to Reelin by exhibiting a sharp increase in tyrosine phosphorylation of Dab1. Western analysis using anti-Dab1 and anti-NSE antibodies confirmed equal sample loading. Thus, these results demonstrate that Cdk5 activity is not required for Reelin-induced tyrosine phosphorylation of Dab 1.

Serine Phosphorylation of Dab1 Occurs Independently of Reelin

Since Reelin-induced tyrosine phosphorylation of Dab 1 was not affected in Cdk5^{-/-} neurons, we cannot place Cdk5 upstream of Reelin in a linear signaling pathway. Therefore, we investigated the possibility that Cdk5-mediated serine phosphorylation of Dab1 lies downstream of Reelin. Western analysis, using anti-phosphotyrosine and anti-Pser491 antibodies, was carried on Dab1 immunoprecipitated from brain extracts prepared from E14.5 reeler or wild type embryos. As observed previously, Dab1 levels were significantly elevated in reeler brains, whereas tyrosine phosphorylation was barely detectable (Rice et al., Development 125:3719-3729, 1998; Howell et al., Genes Dev 13:643-648, 1999). In contrast, there was no apparent change in the stoichiometry of phosphorylation of serine 491 in reeler. To further examine the effect of Reelin stimulation on serine phosphorylation of Dab 1, neurons isolated from E16.5 reeler brains were treated in vitro with exogenous Reelin for 15 minutes at 37° C. As expected, Reelin stimulation resulted in a sharp increase in tyrosine phosphorylation of Dab1. In contrast, serine 491 was constitutively phosphorylated in these neurons, and exposure to Reelin did not cause any noticeable change. These results suggest that Cdk5-mediated serine phosphorylation of Dab1 occurs independently of Reelin.

Phosphorylation of Dab1 During Development

To gain some understanding of the role of Cdk5-mediated serine phosphorylation of Dab1 during the course of brain development, we compared serine and tyrosine phosphorylation of Dab1 in E15.5 embryonic and in postnatal P0, P3, P8, P16, and P42 brains. Dab1 was phosphorylated on tyrosine only in the embryonic and early postnatal brains, and no significant tyrosine phosphorylation of Dab1 was observed after post-natal day 16. In contrast, phosphorylation of serine 491 was present in both embryonic and adult tissue. Indeed, serine phosphorylation of Dab1 closely matched the pattern of Cdk5 expression and activity, which has been shown to be very low during embryogenesis but to peak in the adult (Tsai et al., Development 119:1029-1040, 1993).

Discussion

Reelin and Cdk5 have emerged as critical components of two signaling pathways that regulate neuronal migration during mammalian brain development (Rice and Curran, Annu Rev Neurosci 24:1005-1039, 2001). Mice with disruptions in reelin, dab1, or both vldlr and apoER2 exhibit the reeler phenotype, which led to the placement of these molecules within a signaling pathway (D'Arcangelo et al., Nature 374:719-723, 1995; Howell et al., Nature 389:733-737, 1997; Sheldon et al., Nature 389:730-733, 1997; Trommsdorff et al., Cell 97:689-701, 1999). The lamination defects observed in mice deficient in either Cdk5 (Ohshima et al., Proc Natl Acad Sci USA 93:11173-11178, 1996; Gilmore et al., J Neurosci 18:6370-6377, 1998; Ohshima et al., J Neurosci 19:6017-6026, 1999), p35 (Kwon and Tsai, J Comp Neurol 395:510-522, 1998), or both p35 and p39 (Ko et al., J Neurosci 21:6758-6771, 2001) are less severe but similar to those found in reeler, suggesting that there may be a crosstalk between the Reelin and Cdk5 pathways. Therefore, we hypothesized that such a biochemical link between the two pathways might involve direct phosphorylation of Dab1 by Cdk5. Here we show that Cdk5 phosphorylates Dab1 on serine 491 in vivo.

Our findings present an interesting possibility that Dab1 may serve as a point of convergence for the Reelin and Cdk5 pathways. Dab1 contains several features of an adapter molecule that may be involved in more than one pathway. The PTB domain within its amino terminal region anchors Dab1 to NPxY sequence motifs present within the cytoplasmic domains of various receptors, including the lipoprotein receptors VLDLR and ApoER2 (Trommsdorff et al., J Biol Chem 273:33556-33560, 1998), and the amyloid peptide precursor (APP) family of proteins (Homayouni et al., J Neurosci 19:7507-7515, 1999; Howell et al., Mol Cell Biol 19:5179-5188, 1999). Dab1 contains several tyrosine residues that are critical for its function in the Reelin pathway (Howell et al., Curr Biol 10:877-885, 2000), and at least two of these tyrosines are phosphorylated in response to Reelin stimulation (Keshvara et al., J Biol Chem 276:16008-16014, 2001). Reelin-induced phosphorylation of these tyrosines is likely to result in creation of docking sites on Dab1 for recruitment of signaling molecules containing Src homology 2 (SH2) domains (Songyang et al., Cell 72:767-778, 1993). At least in vitro, tyrosine phosphorylated Dab1 has been shown to interact with SH2 domain-containing proteins (Howell et al., EMBO J 16:121-132, 1997). Therefore, Dab1 is likely to serve as a scaffold for assembly of a signaling complex that would couple an upstream signal from Reelin to the molecular machinery involved in cell positioning. Cdk5-mediated serine phosphorylation could potentially modulate the function of the Dab1-associated signaling complex. For example, it is possible that phosphorylated serine residues could serve to recruit additional signaling proteins containing phosphoserine protein-interaction modules, similar to the phosphoserine binding exhibited by 14-3-3 proteins (Yaffe and Elia, 2001). Alternatively, phosphorylation on serine may induce a conformational change in Dab1 that might influence its interaction with downstream effector molecules.

Unlike tyrosine phosphorylation of Dab1, phosphorylation of serine 491 by Cdk5 occurs independently of the presence of Reelin. Therefore, Reelin is unlikely to play a role in regulating Cdk5 activity. Conversely, Cdk5-deficient neurons exhibit a normal Reelin response, whereby Dab1 is tyrosine phosphorylated as a result of exposure to Reelin. Therefore, Cdk5-mediated serine phosphorylation of Dab 1 is not required for the Reelin-response. Also, examination of brain lysates from different ages showed that tyrosine phosphorylation of Dab1 is restricted to embryonic and early post-natal brains, whereas serine phosphorylation of Dab1 occurs throughout development and in the adult. Taken together, these results are consistent with a model in which Reelin and Cdk5 are components of two distinct signaling pathways. Indeed, recent analysis of mice lacking both p35 and Dab1 revealed additive defects in the cerebellum and in the hippocampus, suggesting that neuronal positioning is regulated by contributions from both of these pathways (Ohshima et al., Proc Natl Acad Sci USA 98:2764-2769, 2001). Nevertheless, a cross-talk between the two pathways, through serine phosphorylation of Dab1, may represent an important mechanism for fine tuning of neuronal migration.

Persistence of serine phosphorylation of Dab1 in adult mice suggests a potential role for Cdk5-mediated Dab1 phosphorylation in neuronal functions that occur after the completion of cell positioning. Indeed, the Reelin signaling pathway has been implicated in synaptogenesis (Borrell et al., J Neurosci 19:1345-1358, 1999; Rice et al., Neuron 31:929-941, 2001). Therefore, it is possible that serine phosphorylation of Dab1 may play a role in such post-positioning functions. It is not known if Dab1 is involved in other cellular processes. However, in light of our results indicating a biochemical link between Cdk5 and Dab 1, it will now be important to investigate a potential role for Dab1 in events that require Cdk5. Cdk5 is thought to contribute to the pathology of neurodegerative disorders, such as Alzheimer's disease (Patrick et al., Nature 402:615-622, 1999) and amyotrophic lateral sclerosis (ALS) (Nguyen et al., Neuron 30:135-147, 2001). Cdk5 is also implicated in the regulation of numerous other cellular events including neurite extension (Nikolic et al., Genes Dev 10:816-825, 1996), cell adhesion (Homayouni and Curran, Curr Biol 10:R331-334, 2000; Kwon et al., Curr Biol 10:363-372, 2000) and axonal transport (Niethamrnmer et al., Neuron 28:697-711, 2000; Sasaki et al., Neuron 28:681-696, 2000; Wynshaw-Boris and Gambello, Genes Dev 15:639-651, 2001). Therefore, given the overlap of the Cdk5 and Dab1 expression patterns, it is possible that Dab1 may also participate in these cellular processes.

Dab1 was found to accumulate in a hypophosphorylated form in Cdk5^{-/-} neurons. Decreased tyrosine phosphorylation of Dab1 and its subsequent accumulation were very slight at E16.5 in Cdk5^{-/-} brains, but these changes became dramatic by P0. One possible interpretation of this result is that Cdk5^{-/-} neurons suffer from an intrinsic biochemical defect that prevents Reelin-induced Dab1 tyrosine phosphorylation. However, this is unlikely, because cultured Cdk5^{-/-} neurons exhibited a normal Dab 1 tyrosine phosphorylation response when treated with exogenous Reelin. When Dab1 expression was analyzed by immunohistochemistry, Dab1 accumulation was particularly prevalent in neurons located in deeper layers of the cerebral cortex and the cerebellum. Previous neuroanatomic analysis of Cdk5- and p35-deficient mice demonstrated that the first cohorts of neurons are able to migrate past subplate neurons, but later-born neurons fail to overtake their predecessors (Gilmore et al., J Neurosci 18:6370-6377, 1998; Kwon and Tsai, J Comp Neurol 395:510-522, 1998). These subsequent failures in migration lead to ectopic accumulation of neurons beneath the subplate. Similarly, in the cerebellum, Purkinje cells fail to migrate outward to form the PCL, and instead they are clustered in deep regions of the cerebellum (Ohshima et al., J Neurocsi 19:6017-6026, 1999). The accumulation of Dab1 in Cdk5^{-/-} mice is confined to these ectopic neurons arrested deep within the cortex and the cerebellum. The most straightforward interpretation of our results is that in the absence of Cdk5, ectopic neurons fail to reach the source of Reelin, which is expressed in the marginal zone of the cortex and in the external germinal layer and nuclear transitory zone of the cerebellum in the developing brain. This effect becomes progressively more severe at later stages of development when neurons have to traverse greater distances to contact Reelin. Therefore, it is unlikely that the diminished Reelin signaling in Cdk5^{-/-} neurons during late embryogenesis is due to an intrinsic defect in the Reelin pathway within these neurons, but rather it is a consequence of their ectopic location. This hypothesis can be tested by expressing Reelin in locations more proximal to the ectopic neurons of the cerebral cortex and cerebellum in Cdk5^{-/-} mice.

We identified serine 491 of Dab1 as a Cdk5-dependent site of in vivo phosphorylation. However, we cannot rule out the possibility that there are other Cdk5 phosphorylation sites in Dab1. Indeed, our phosphopeptide mapping analysis shows that Cdk5 also phosphorylates serine 515 of Dab1 in vitro. Nevertheless, antibodies against phosphoserine 491 of Dab1 provide a valuable marker for Cdk5 activity in vivo, and they can be used to further investigate the role of Cdk5 in neural functions within cells that express Dab1. While the functional significance of Cdk5-catalyzed phosphorylation of Dab1 is not yet clear, our results suggest that Reelin and Cdk5 are components of two distinct signaling pathways that may converge on Dab1. Cdk5-mediated serine phosphorylation of Dab1 may serve to modulate signaling events initiated by Reelin that lie downstream of Dab1 tyrosine phosphorylation. Alternatively, serine phosphorylation might point to a potential role for Dab1 beyond the Reelin signaling pathway.

We claim:

1. A method for detecting cyclin dependent kinase 5 (Cdk5) activity in a biological sample, which method comprises determining whether Disabled 1 protein (Dab1) in said sample is phosphorylated on a candidate sequence preferred by cdk 5 activity, wherein phosphorylation of Dab1 on said candidate sequence indicates the presence of active Cdk5 in said sample.

2. The method of claim 1 wherein the candidate sequence contains the Cdk5 amino acids consisting of serine 491 and 515.

3. The method of claim 1 wherein the candidate sequence is selected from the group consisting of tryptic peptides QSSPSK (SEQ ID NO:1) and SSASHVSDPTADDIFEEGFESPSK (SEQ ID NO:2).

4. The method of claim 1 wherein said biological sample is derived from an organism selected from the group consisting of mouse and human.

5. The method of claim 1 wherein said biological sample is derived from the group consisting of brain and blood.

6. The method of claim 1 wherein said biological sample is derived from a cell culture.

7. The method of claim 1 wherein said Dab1 phosphorylation occurs in vivo.

8. The method of claim 1 which comprises immunoprecipitating said Dab1 from said biological sample prior to said determining step using an antibody that binds to phosphorylated and unphosphorylated Dab1.

9. The method of claim 1 which comprises immunoprecipitating said Dab1 from said biological sample prior to said determining step using an antibody that binds to Dab1 only when it is phosphorylated on a Cdk5 candidate sequence.

10. The method of claim 1 wherein Dab1 phosphorylation is determined using an antibody that binds to Dab1 only when it is phosphorylated on a candidate sequence preferred by Cdk5 activity.

11. The method of claim 10 wherein said antibody is raised against the polypeptide fragment TPAPRQSS(PO₄)PSKSSA (SEQ ID NO:3 which contains a phosphate group on serine 491).

12. The method of claim 10 wherein said antibody detects Dab1 phosphorylation on amino acids consisting of serine 491 and serine 515.

13. The method of claim 10 wherein said antibody is polyclonal.

14. The method of claim 10 wherein said antibody is monoclonal.

15. The method of claim 10 wherein Dab1 phosphorylation is determined by using techniques consisting of radioimmunoassay, ELISA, “sandwich” immunoassays, immunoradiometric assays, gel diffusion precipitation reactions, immunodiffusion assays, in situ immunoassays, western blots, precipitation reactions, agglutination assays, complement fixation assays, immunofluorescence assays, protein A assays, immunoelectrophoresis assays, mass spectrometry and antibody array.

16. A method for identifying a compound that inhibits or decreases Cdk5 activity which could be used for the treatment of a disease or condition where Cdk5 activity is involved in pathogenesis, which method comprises
(a) determining whether Dab1 in a test sample is phosphorylated on a candidate sequence preferred by Cdk5 activity in the presence of said compound and active Cdk5 and
(b) comparing Cdk5 activity in said test sample with Cdk5 activity in a control sample which contains active Cdk5 but lacks the compound, wherein decreased phosphorylation of Dab 1 in said test sample as compared to said control sample indicates a compound that is capable of inhibiting or decreasing Cdk5 activity.

17. The method of claim 16 wherein said condition is selected from the group consisting of Parkinson's disease, Huntington's disease, Alzheimer's disease, amyotrophic lateral sclerosis (ALS), stroke, epilepsy, lissencephaly and trauma.

18. A method for identifying a compound that increases Cdk5 activity which could be used for the treatment of a disease or condition in which Cdk5 activity is involved in pathogenesis, which method comprises
(a) determining whether Dab1 in a test sample is phosphorylated on a candidate sequence preferred by Cdk5 activity in the presence of said compound and active Cdk5 and
(b) comparing Cdk5 activity in said test sample with Cdk5 activity in a control sample which contains active Cdk5 but lacks the compound, wherein increased phosphorylation of Dab1 in said test sample as compared to said control sample indicates a compound that is capable of increasing Cdk5 activity.

19. The method of claim 18 wherein said condition is selected from the group consisting of Parkinson's disease, Huntington's disease, Alzheimer's disease, amyotrophic lateral sclerosis (ALS), stroke, epilepsy, lissencephaly and trauma.

20. A method for treating Alzheimer's disease and ALS comprising administering a therapeutically effective amount of a compound which is identified using the method of claim 16.

21. A method for treating epilepsy and lissencephaly comprising administering a therapeutically effective amount of a compound which is identified using the method of claim 18.

22. A method for detecting neurological disorders in a subject, which method comprises detecting Cdk5 activity in said subject according to the method of claim 1 wherein a decrease of said Dab1 phosphorylation in said biological sample as compared to a control biological sample from a control subject indicates the presence of a neurological disorder.

23. A method for detecting neurological disorders in a subject, which method comprises detecting Cdk5 activity in said subject according to the method of claim 1 wherein an increase of said Dab1 phosphorylation in said biological sample as compared to a control biological sample from a control subject indicates the presence of a neurological disorder.

24. A method for detecting neurological disorders, which method comprises detecting Cdk5 activity according to the method of claim 12 wherein a decrease or lack of said Dab1 phosphorylation in said biological sample as compared to a control biological sample from a control subject indicates the presence of a neurological disorder.

25. A method for detecting neurological disorders, which method comprises detecting Cdk5 activity according to the method of claim 12 wherein an increase of said Dab1 phosphorylation in said biological sample as compared to a control biological sample indicates the presence of a neurological disorder.

26. A screening kit comprising
(a) an antibody that binds to Dab1 only when it is phosphorylated on a candidate sequence preferred by Cdk5 activity; and
(b) reagents suitable for detecting the binding of said antibody to Dab 1.

27. The screening kit of claim 26 wherein said reagents comprise reagents used in techniques consisting of radioimmunoassay, ELISA, “sandwich” immunoassays, immunoradiometric assays, gel diffusion precipitation reactions, immunodiffusion assays, in situ immunoassays, western blots, precipitation reactions, agglutination assays, complement fixation assays, immunofluorescence assays, protein A assays, immunoelectrophoresis assays, mass spectrometry and antibody array.

28. An antibody that binds to Dab1 only when it is phosphorylated on a candidate sequence preferred by Cdk5 activity.

29. The antibody of claim 28 which is raised against the polypeptide fragment TPAPRQSS(PO₄)PSKSSA (SEQ ID NO:3 which contains a phosphate group on serine 491).

30. The antibody of claim 28 that detects Dab1 phosphorylation on amino acids consisting of serine 491 and serine 515.

31. A method for quantitating the level of cyclin dependent kinase 5 (Cdk5) activity in a biological sample, which method comprises
(a) determining the amount of Disabled 1 protein (Dab1) in said sample which is phosphorylated on a candidate sequence preferred by Cdk 5 activity, and
(b) determining the total amount of Dab1 in said sample, wherein the proportion of Dab1 which is is phosphorylated on said candidate sequence represents a quantitative measure of the level of Cdk5 activity in said sample.
